(19) United States
(12) Patent Application Publication
(10) Pub. No.: US 2021/0393429 A1
(43) Pub. Date: Dec. 23, 2021

(54) FLEXIBLE WRIST ORTHOSIS

(71) Applicant: MILLET INNOVATION, Loriol Sur Drome (FR)

(72) Inventor: Maria Luisa TREPIER-LE BELLER, Portes-Les-Valence (FR)

(73) Assignee: MILLET INNOVATION, Loriol Sur Drome (FR)

(21) Appl. No.: 17/292,091

(22) PCT Filed: Nov. 5, 2019
(86) PCT No.: PCT/FR2019/052615

§ 371 Date: May 7, 2021

(30) Foreign Application Priority Data
Nov. 7, 2018 (FR) 18 60254

(51) Int. Cl. A61F 5/01

(52) U.S. Cl. CPC A61F 5/0118

(57) ABSTRACT

A hand and wrist orthosis including: a sleeve having a proximal portion shaped to match the part of the forearm extending to the wrist, and a distal portion shaped to match the part of the hand extending from the base of the metacarpophalangeal joints of the fingers to the wrist, the distal portion having a first opening for the passage of the thumb, a second opening for the passage of the other fingers, the proximal portion shaped to conform to the wrist and a portion of the forearm covered by the sleeve, the sleeve shaped to constrain the wrist against a bending movement toward the palmar side of the hand, the sleeve and a support band being made of an elastic material; attached to the distal and proximal portions of the sleeve and adapted to cover only an upper part of the narrower region between opposite sides of the wrist.

The present invention relates to an orthosis for a joint and more particularly the wrist joint.

For decades, orthopedics has been devoted to pathologies requiring restraint and/or immobilization. For some years now, it has been endeavored to spare the joints as to all their components, without impeding their usual use. For example, for osteoarthritis of the thumb, the Applicant has developed a light orthosis that minimizes involuntary movements of the thumb by applying mechanical principles (patent EP 3 041 441).

However, it appeared that such a light orthosis could be beneficial not only for relieving pain related to osteoarthritis, but also for a pathology whose occurrence is just as important as for osteoarthritis of the thumb. This is carpal tunnel syndrome, whose high frequency of occurrence is due to the multiplicity of risk factors and their combined effect. In all cases, the aim is to limit the pressure on the median nerve in the carpal tunnel, through which the flexor tendons of the hand and their synovial sheaths pass. Carpal tunnel syndrome is characterized by sensations of tingling, pins and needles, numbness or electrical discharges mainly located on the palmar surface of the first three fingers (anatomical territory of the median nerve) and by a loss of muscle strength in the affected wrist and hand. These discomforts are often felt at night, waking the patient. They can also appear during the day, triggered by certain movements or by holding the hand in certain harmful positions.

It appears that certain movements and postures favor the appearance of carpal tunnel syndrome, its persistence and/or its aggravation. A simple solution to relieve and limit the evolution, or even resolve this condition consists in limiting the frequency and amplitude of these hazardous movements and postures in everyday life. Certain gestures are particularly restrictive for the median nerve involved in carpal tunnel syndrome. In particular, extreme angulations of the wrist have the effect of compressing the carpal tunnel acutely against the annular ligament of the carpus during flexion (palmar side of the hand) and against the carpal bones during extension (dorsal side of the hand). The median nerve may also be chronically compressed, often as a result of inflammation of the tendons of the flexor muscles of the fingers and/or their synovial sheaths and their volume increase.

Carpal tunnel syndrome is usually treated first by wearing a wrist splint at night and/or by infiltrations (cortisone injections into the carpal tunnel). The splints that are generally used immobilize the wrist joint in a neutral position, which avoids postures that can significantly compress the median nerve. The main disadvantage of these splints is their rigidity, which prevents normal use of the hand and wrist. Consequently, they are very uncomfortable, and it is harmful for the patient to wear such a splint in everyday activities requiring hand work. Compensations on other joints as well as abnormally high efforts would then be necessary to carry out these common tasks, which can cause the appearance of secondary pathologies.

It is therefore desirable to provide an orthosis that minimizes the range of motion of the wrist joint during the phases of neutral reflex position, and that allows the full range of motion of the joint during the phases of voluntary movement related to current activities, in all possible axes of rotation. It may also be desirable to dampen voluntary movements, especially to favor the return to the neutral position.

Embodiments relate to a hand and wrist orthosis comprising: a sleeve having a distal portion and a proximal portion, the proximal portion being shaped to match the shape of a part of the forearm extending to the wrist, the distal portion being shaped to match the shape of a part of the hand extending from the base of the metacarpophalangeal joints of the fingers to the wrist, the distal portion having a first opening for the passage of the thumb, a second opening for the passage of the other fingers, the proximal portion being shaped to conform to the shape of the wrist and a portion of the forearm covered by the sleeve, the sleeve being shaped to constrain the wrist against a bending movement toward the palmar side of the hand, the sleeve being made of an elastic material; and a support band made of an elastic material, attached to the distal and proximal portions of the sleeve and adapted to cover only an upper part of the narrower region of the wrist between opposite sides of the wrist.

According to an embodiment, the sleeve is made from a single piece including a dorsal portion adapted to cover the dorsal portion of the hand and wrist, a palmar portion adapted to cover a palmar portion of the hand and wrist, and a band connecting the dorsal and palmar portions.

According to an embodiment, the sleeve has a slit formed in the dorsal portion between the proximal and distal portions of the sleeve, and extending between two opposite edges of the dorsal portion, the opposite edges of the slit being attached to each other to close the slit, the support band being attached to the slit when closed.

According to an embodiment, the slit is spindle-shaped by removal of a portion of the material forming the sleeve, or is formed without removal of material forming the sleeve, wherein the opposing edges of the slit are secured to each other to provide a spindle-shaped overlap area of the material forming the sleeve.

According to an embodiment, the sleeve is assembled by two attachment lines that are positioned on lateral regions of the wrist and hand, each of the attachment lines connecting an edge of the dorsal part with a longer edge of the palmar part.

According to an embodiment, the two attachment lines comprise an attachment line adapted to extend along an outer lateral side of the hand passing distally over the dorsal side of the hand.

According to an embodiment, the first opening of the sleeve for the passage of the thumb has a notch formed in a dorsal part of the sleeve in the prolongation of the thumb so as to favor lateral movements of the thumb.

According to an embodiment, a dorsal part of the sleeve comprises a fold extending from the first opening in the prolongation of the thumb to favor free lateral movements of the wrist and opening of the hand.

According to an embodiment, the sleeve is made in a plurality of sizes so that a distal edge of the sleeve is stretched transversely by at most 20% and the proximal portion of the sleeve is stretched transversely by at most 10%.

According to an embodiment, the material forming the sleeve has at least one of the following features: the material has a Young's modulus along the longitudinal axis of the sleeve between 0.5 MPa and 1 MPa; and the material has a thickness between 1 mm and 1.4 mm.

According to an embodiment, the material forming the sleeve comprises, over its entire extent, two layers of elastic fabric assembled by gluing.

According to an embodiment, each of the two fabric layers is a fabric having at least one of the following features: the fabric comprises between 75 and 85% by weight of polyamide and between 15 and 25% by weight of elastane; and the fabric has an elasticity of 85 to 115% in the warp direction and 65 to 95% in the weft direction, wherein the warp of the fabric is oriented axially on the dorsal portion of the sleeve.

According to an embodiment, the support band is made of an elastic material similar to the material forming the sleeve.

According to an embodiment, the support band has at least one of the following features: the support band is connected to the sleeve by a zigzag seam so as to favor elastic extension of the orthosis in the longitudinal direction of the support band; the support band has a width between 0.3 and 1.5 cm; the support band covers between 30 and 70% of the circumference of the wrist; the support band is attached to the sleeve only at one end and secured at another end by a tightening device to adjust the tension of the support band around the wrist; and the support band is made of an elastic fabric coated with a layer of a polymer gel.

According to an embodiment, the orthosis comprises a device for tightening the sleeve around the forearm.

Exemplary embodiments of the invention will be described in the following in a non-limiting manner in relation to the attached figures among which:

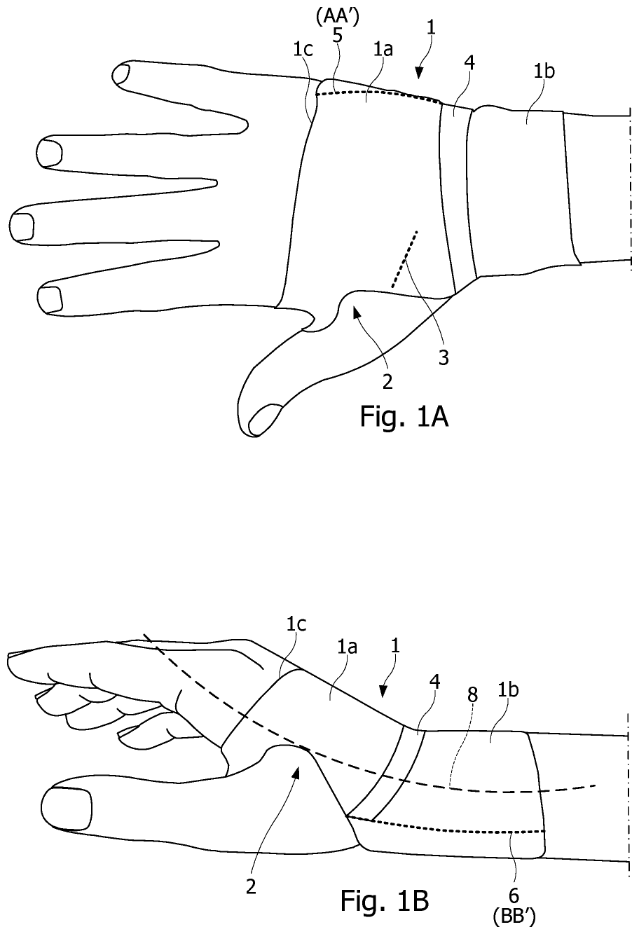

FIGS. 1A and 1B are top and side views, respectively, of a hand wearing an orthosis according to an embodiment.

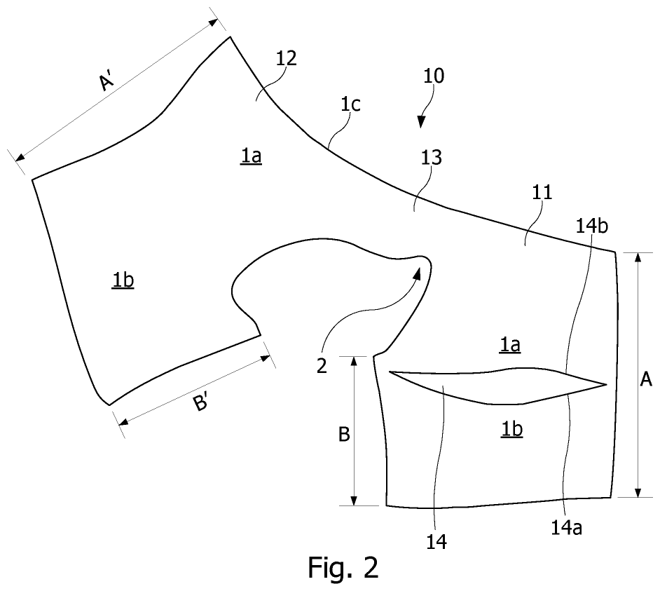

FIG. 2 illustrates the shape of a thin component that is assembled to form the orthosis of FIG. 1A, according to an embodiment.

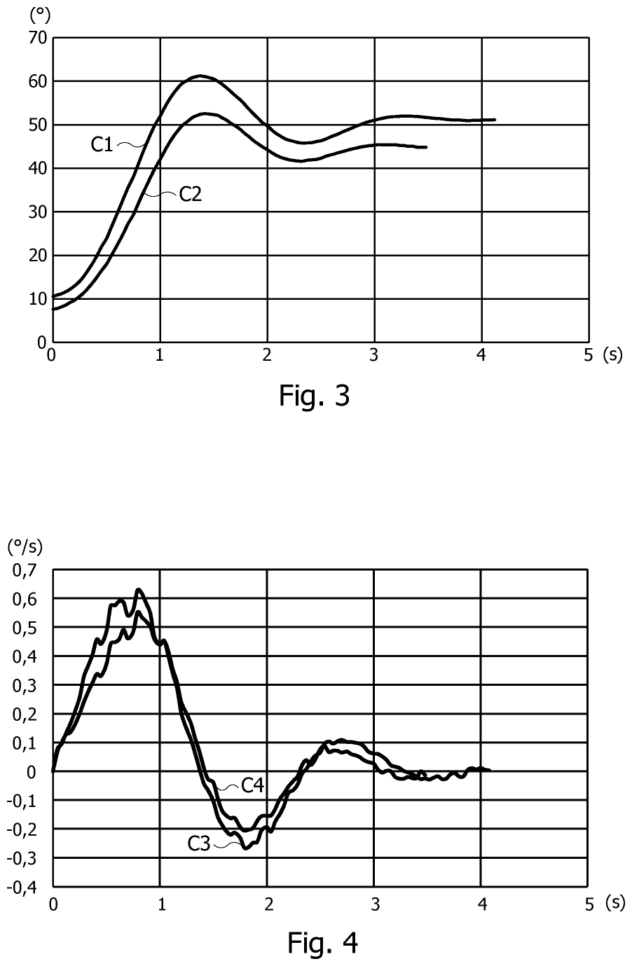

FIG. 3 illustrates time diagrams of wrist movements, with and without the orthosis,

FIG. 4 illustrates time diagrams of wrist speeds of movement, with and without the orthosis.

FIGS. 1A and 1B show an orthosis according to an embodiment. The orthosis comprises a sleeve 1 covering the wrist and a portion of the forearm and extending to the origin of the finger joints. The sleeve includes a distal portion 1a and a proximal portion 1b, and each of these portions includes a dorsal portion and a palmar portion. The distal portion 1a of the sleeve is shaped to conform to the shape of the portion of the hand extending from the base of the metacarpophalangeal joints of the fingers to the edge of the wrist. The distal portion 1a has a distal edge 1c forming a first opening for the passage of the thumb and a second opening for the passage of the other fingers, namely the index, middle, ring, and little fingers. The proximal part 1b is shaped to match the shape of the wrist and the part of the forearm covered by the orthosis 1.

A functional notch 2 may be made in the dorsal part of the portion 1a of the sleeve 1, at the level of the thumb. The notch 2 allows free movement of the thumb and lateral movements of the wrist, especially abduction.

A functional fold 3 may be deliberately positioned on the dorsal part of the sleeve 1, starting from the opening for the passage of the thumb, in the prolongation of the thumb, in order to not hinder lateral movements of the wrist and the opening of the hand.

FIG. 2 illustrates a component 10 made of a thin elastic material, which is assembled to form the sleeve 1, according to an embodiment. The component 10 comprises a dorsal portion 11, and a palmar portion 12 connected between them by a band 13. The dorsal portion 11 and the palmar portion 12 are shaped to cover and conform to the shape of the dorsal portion of the hand, the wrist and a portion of the forearm, respectively, and the shape of the palmar portion of the hand, the wrist and the forearm, respectively. The back portion 11 of the component 10 has a spindle-shaped slot 14 extending between joining edges A and B of the back portion 11. The slot 14 is formed in the area of the back portion 11 intended to cover the wrist.

The palmar portion 12 of the component 10 has assembly edges A′, B′. The assembly edges A and A′ are intended to be secured together and to lie along an outer lateral region of the portion covered by the sleeve 1, of the wrist, forearm and hand. Connecting edges B and B′ are adapted to be secured together and to extend along an inner lateral region of the portion covered by the sleeve 1, from the forearm, wrist, to the base of the thumb.

The band 13 of the component 10 is shaped to cover the hand between the dorsal and palmar regions of the hand through the fold region between the thumb and index finger. The indentation 2 is achieved in the band 13.

The edges 14a, 14b of the slot 14 are joined edge to edge. In addition, the edges A and B of the component 10 are shorter than the edges A′ and B′. These two arrangements increase the ergonomics and comfort of the orthosis 1. Indeed, they give it a curve 8 that fits the shape of the hand by constraining it against the flexing movement on the side of the palmar face of the hand.

The external lateral seam joining the edges A, A′ of the orthosis 1 can be deliberately offset distally towards the back of the hand to minimize the efforts of lateral wrist movements, particularly abduction. The finish of this seam is preferably flat and non-aggressive to guarantee comfort when worn.

By making the sleeve 1 from a single piece (10), it is possible to avoid the presence of a seam in the vicinity of the fold region between the thumb and the index finger, as such a seam inevitably produces a rigidity that can hinder the movements of the thumb and in particular the gripping function of the hand. However, it is possible to make the sleeve from two parts and to assemble these two parts, in particular with a seam practiced at a location where it is likely not to cause discomfort, for example by cutting the component 10 in the prolongation of the notch 2, and/or in the dorsal part 11.

A support band 4 is positioned opposite the wrist and runs halfway around it on the dorsal side of the hand. The support band 4 thus covers the seam closing the slot 14 and ensures the correct positioning of the orthosis between the hand and the forearm. It also optimizes a progressive support of the wrist in flexion without imposing significant pressure on the carpal tunnel zone while allowing wrist extension with the minimum of constraints.

Indeed, the addition of the support band 4 increases the rigidity of the orthosis in a very specific area. When the wrist flexes on the palmar side of the hand, the wrist circumference increases. However, this increase in the circumference of the wrist is not evenly distributed around the wrist. Indeed, the arc described by the dorsal face of the wrist increases in length while the arc described by the palmar face of the wrist decreases in length. Thus, the support band 4 is positioned along the arc of the dorsal face of the wrist at the thinnest region of the wrist, i.e. just above the styloid protuberance on the forearm side and just below the carpo-metacarpal joint of the thumb on the hand side. The support band 4 thus induces an increase in the resistance of the orthosis 1 to the deformation of the wrist along the band 4 and thus an increase in the tension on the band 4 that counteracts the flexion movement. On the other hand, nothing is added on the palmar side of the wrist, which therefore includes only a single thickness of the material from which the component 10 is formed, so that the orthosis can absorb the deformations without imposing a significant increase in pressure with respect to the carpal tunnel zone.

Furthermore, because the support band 4 is positioned around the narrowest region of the wrist, the orthosis is ideally positioned and can only move axially with difficulty towards the hand or forearm, in the absence of external assistance. Thus, the support band 4 creates a first fixed point under the carpo-metacarpal joint and stiffens the orthosis on the upper part of the narrowest region of the wrist. The positioning of the orthosis is also limited by the band 13 passing over the fold zone between the thumb and the index finger, preventing the orthosis from moving towards the forearm, and thus constituting a second fixed point. However, the distance between the fold area of the thumb and the narrowest area of the wrist increases with wrist flexion. The support band 4 therefore requires that the longitudinal elongation of the dorsal face of the sleeve 1 accompanying the wrist flexion movement on the side of the palmar face of the hand be carried out between these two fixed points on the dorsal part. The length of the orthosis over which the deformation takes place is thus limited, which increases the resistance of the orthosis to the wrist flexion movement. These two phenomena are added to the cantilever effect of the orthosis covering the limbs on either side of the joint to generate a moderate but meaningful resistance to wrist flexion, thus limiting the amplitude and speed of non-voluntary wrist flexion movements and providing a feeling of support during voluntary movements without impeding them. This restriction of the flexion movement is achieved by minimizing the restriction of the movement within a comfort angle range. According to the ergonomics and manual handling standard ISO 11228-3, the comfort flexion angle of the wrist does not exceed 45° from a neutral position.

The resistance that the orthosis opposes to the wrist extension movement (on the dorsal side of the hand) is minimized by its configuration. Indeed, the cantilever effect is always present in antagonism to this extension movement, but the palmar side of the orthosis does not present any localized stiffening (no support band). Thus, the deformation induced by the wrist extension is absorbed over a much greater length (in the axial direction of the sleeve 1). In addition, the support band 4 does not prevent the fabric from wrinkling on the dorsal side of the orthosis to absorb the decrease in length, due to its small width and its positioning along the wrist contour and only on the dorsal side of the sleeve 1.

Thus, the support band 4 exerts a significant resistance to a wrist flexion movement (on the palmar side of the hand), without increasing the resistance to a wrist extension movement (on the dorsal side of the hand), this last movement being extremely sensitive to stress. The pressure exerted by the orthosis on the carpal tunnel area is also minimized.

FIG. 3 illustrates time diagrams C1, C2 of uncontrolled flexion movements of the hand under the effect of gravity, the hand being initially placed substantially horizontally, the forearm being held fixed in the natural extension of the hand. The amplitude of the hand flexion movement is measured by the flexion angle. The curves C1, C2 were obtained respectively in the absence and presence of the orthosis.

Curves C1, C2 each have a first peak and a first dip. By comparing the flexion angles corresponding to these first peaks and first dips, it appears that wearing the orthosis (curve C2) leads to a decrease in the flexion angle of the first peak of approximately 10.1° (i.e. 16.5%). The flexion angle at the first dip is also decreased by about 5.6° (i.e. 12.2%). A decrease can also be observed in the differential between the maximum and minimum amplitude of about 29.2% and a decrease in the angle of flexion once the movement is completed of about 7.7° (i.e. 15%), the final position being reached more quickly with the orthosis. In addition, wrist movements appeared to stabilize more quickly with the orthosis on. Indeed, the flexion angle stabilizes at about 3.4 s with the orthosis and at about 3.8 s without the orthosis.

It may be noted that the starting points of the curves C1, C2 are different, with the starting point of the curve C2 being about 3° above that of the curve C1. This means that the hand starts from a higher initial position in the case of curve C2 than in the case of curve C1, which would normally tend to increase at least the value of the first peak of curve C2 and thus minimize the differences in angular position between curves C1 and C2. This difference in initial position also shows the pre-stressing effect exerted by the orthosis against wrist flexion movements, which thus tends to straighten the hand.

FIG. 4 illustrates time diagrams C3, C4 of the variation in angular velocity of uncontrolled flexion movements of the hand under the effect of gravity, with the hand initially placed substantially horizontally, the forearm being held fixed in the natural extension of the hand. Curves C3, C4 were obtained respectively in the absence and presence of the orthosis. Curves C3, C4 show a first peak of speed corresponding to the fall of the hand, then a passage through a zero-speed point corresponding to the position of the wrist in maximum flexion after the fall. Then, a first dip appears corresponding to the rebound of the hand in the opposite direction of the fall. The following peaks and dips correspond to the subsequent oscillations of lesser amplitude and damping. Comparing the curves C3 (without orthosis) and C4 (with orthosis), the velocities corresponding to the first peak and the first dip are reduced thanks to the orthosis, respectively by about 13% for the first peak and by about 26% for the first dip. It can also be seen that the gap between the first peak and the first dip is significantly reduced by about 17%, thanks to the orthosis. It can also be observed that the times between these peaks and dips are approximately the same with and without the orthosis (about 1 s), which implies that the orthosis also reduces the angular acceleration in a significant manner.

The analysis of these measurements also shows that wearing the orthosis allows to decrease the extreme values of the amplitudes, speeds and accelerations of the passive and unconscious movements of the wrist, and this without hindering the voluntary movements.

In an embodiment, the sleeve 1 is shaped to exert limited compression on the underlying tissues of the hand, so as not to cause secondary pathologies or discomfort. This result is achieved by providing several sizes of the sleeve in order to limit the transverse stretch of the distal edge 1a of the sleeve between 0 and 20% and preferably between 2.5 and 17.5%. The choice of size is made by measuring the circumference of the hand in the region covered by the distal part 1a of the sleeve. Furthermore, the circumference of the sleeve 1 around the narrowest region of the wrist has been dimensioned for the different sizes so that the transverse elongation of the proximal part 1b of the sleeve remains less than 10%, so that it exerts the least compression while remaining in contact with the skin.

According to an embodiment, the component 10 forming the sleeve 1 is made of an elastic material having, along the longitudinal axis of the sleeve, a Young's modulus between 0.5 MPa and 1 MPa.

According to an embodiment, the component 10 forming the sleeve 1 includes over its entire length two layers of elastic fabric assembled by gluing.

According to an embodiment, each of the two layers forming the component 10 is made of a fabric comprising between 75 and 85% by weight of polyamide and between 15 and 25% by weight of elastane.

According to an embodiment, each of the two layers forming the component 10 has a weight of 155 g/m², and/or a thickness between 0.5 and 0.7 mm, wherein either one of these measures provides the user with a reassuring presence of the orthosis.

According to an embodiment, each of the two layers of fabric has an elasticity of 85 to 115% in the warp direction, and 65 to 95% in the weft direction of the fabric, with the warp of the fabric oriented axially on the back of the sleeve.

According to an embodiment, the sleeve 1 is made of a material with a thickness between 1 mm and 1.4 mm.

According to an embodiment, the support band 4 is made of an elastic material having similar mechanical properties to those of the material used for the sleeve 1.

According to an embodiment, the support band 4 is connected to the sleeve 1 by a zigzag seam so as to favor elastic extension of the orthosis in the longitudinal direction of the support band 4.

According to an embodiment, the support band 4 has a width between 0.3 and 1.5 cm.

According to an embodiment, the support band 4 covers between 30 and 70% of the circumference of the wrist.

It will be apparent to a person skilled in the art that the present invention may be subject to various alternatives and applications.

In particular, the invention is not limited to an orthosis for relieving carpal tunnel syndrome, but can be applied to any pathology in which only a pre-stressing of the wrist position in its neutral position is sufficient.

Moreover, other arrangements than the provision of the slot 14, and/or the provision of edges (A-A′, B-B′) of different lengths for the assembled edges of the component 10 may easily be devised to achieve the preload exerted by the orthosis on the wrist joint to maintain the hand in its neutral position relative to the forearm. For example, one or more longitudinal reinforcements of elastic material may be provided along the longitudinal axis of the orthosis. However, these reinforcements should not interfere with voluntary movements of the wrist and hand, as long as these movements are not excessive.

The support band 4 may be made of an elastic material coated with a polymer gel material such as a silicone gel. It may also include one or more branches extending to the distal portion and/or to the proximal portion.

The support band 4 may be attached to the sleeve 1 only by one end and attached in an adjustable manner at its other end, for example by means of a hook and loop device (Velcro®), in order to provide tension adjustment.

Instead of closing the opening 14 by securing the edges 14a, 14b of the opening together, it may be provided to close the opening 14 by means of a piece of another elastic material.

The opening 14 may have been made without removal of fabric so that it is in the form of a slit with parallel edges (when the fabric is laid flat without undergoing stretching), the opening being closed by pulling one of the two edges 14a, 14b toward the other edge to obtain an overlap of portions of the sleeve along the opening 14. A strip may cover this overlap.

A tightening means may also be provided, for example a strap integral with the sleeve associated with a fastening device, for example with hooks and loops, to tighten the proximal part 1b of the sleeve 1 around the part of the forearm covered by the orthosis.

The notch 2 formed in the dorsal edge of the opening of the sleeve for the passage of the thumb may be omitted, bearing in mind that the absence of this feature does not cause any particular inconvenience. The same applies to fold 3.

1. Hand and wrist orthosis comprising:
a sleeve having a distal portion and a proximal portion, the proximal portion being shaped to match the shape of a part of the forearm extending to the wrist, the distal portion being shaped to match the shape of a part of the hand extending from the base of the metacarpophalangeal joints of the fingers to the wrist, the distal portion having a first opening for the passage of the thumb, a second opening for the passage of the other fingers, the proximal portion being shaped to conform to the shape of the wrist and a portion of the forearm covered by the sleeve, the sleeve being shaped to constrain the wrist against a bending movement toward the palmar side of the hand, the sleeve being made of an elastic material; and
a support band made of an elastic material, attached to the distal and proximal portions of the sleeve and adapted to cover only an upper part of the narrower region of the wrist between opposite sides of the wrist.

2. The orthosis according to claim 1, wherein the sleeve is made from a single piece including a dorsal portion adapted to cover the dorsal portion of the hand and wrist, a palmar portion adapted to cover a palmar portion of the hand and wrist, and a band connecting the dorsal and palmar portions.

3. The orthosis according to claim 1, wherein the sleeve has a slit formed in the dorsal portion between the proximal and distal portions of the sleeve, and extending between two opposite edges of the dorsal portion, the opposite edges of the slit being attached to each other to close the slit, the support band being attached to the slit when closed.

4. The orthosis of claim 3, wherein the slit is spindle-shaped by removal of a portion of the material forming the sleeve, or is formed without removal of material forming the sleeve, wherein the opposing edges of the slit are secured to each other to provide a spindle-shaped overlap area of the material forming the sleeve.

5. The orthosis according to claim 1, wherein the sleeve is assembled by two attachment lines that are positioned on lateral regions of the wrist and hand, each of the attachment lines connecting an edge of the dorsal part with a longer edge of the palmar part.

6. The orthosis of claim 5, wherein the two attachment lines comprise an attachment line adapted to extend along an outer lateral side of the hand passing distally over the dorsal side of the hand.

7. The orthosis according to claim 1, wherein the first opening of the sleeve for the passage of the thumb has a notch formed in a dorsal part of the sleeve in the prolongation of the thumb so as to favor lateral movements of the thumb.

8. The orthosis according to claim 1, wherein a dorsal part of the sleeve comprises a fold extending from the first opening in the prolongation of the thumb to favor free lateral movements of the wrist and opening of the hand.

9. The orthosis according to claim 1, wherein the sleeve is made in a plurality of sizes so that a distal edge of the sleeve is stretched transversely by at most 20% and the proximal portion of the sleeve is stretched transversely by at most 10%.

10. The orthosis according to claim 1, wherein the material forming the sleeve has at least one of the following features:
the material has a Young's modulus along the longitudinal axis of the sleeve between 0.5 MPa and 1 MPa, and
the material has a thickness between 1 mm and 1.4 mm.

11. The orthosis according to claim 1, wherein the material forming the sleeve comprises, over its entire extent, two layers of elastic fabric assembled by gluing.

12. The orthosis of claim 11, wherein each of the two fabric layers is a fabric having at least one of the following features:
the fabric comprises between 75 and 85% by weight of polyamide and between 15 and 25% by weight of elastane, and
the fabric has an elasticity of 85 to 115% in the warp direction and 65 to 95% in the weft direction, wherein the warp of the fabric is oriented axially on the dorsal portion of the sleeve.

13. The orthosis according to claim 1, wherein the support band is made of an elastic material similar to the material forming the sleeve.

14. The orthosis of claim 13, wherein the support band has at least one of the following features:
the support band is connected to the sleeve by a zigzag seam so as to favor elastic extension of the orthosis in the longitudinal direction of the support band,
the support band has a width between 0.3 and 1.5 cm,
the support band covers between 30 and 70% of the circumference of the wrist,
the support band is attached to the sleeve only at one end and secured at another end by a tightening device to adjust the tension of the support band around the wrist, and
the support band is made of an elastic fabric coated with a layer of a polymer gel.

15. The orthosis of claim 14, comprising a device for tightening the sleeve around the forearm.
